(19) United States
(12) Patent Application Publication
(10) Pub. No.: US 2019/0336352 A1
(43) Pub. Date: Nov. 7, 2019

(54) Fluid Absorber and Method of Making Same

(71) Applicant: Robert Christmas, Oak Park, IL (US)

(72) Inventor: Robert Christmas, Oak Park, IL (US)

(21) Appl. No.: 15/969,208

(22) Filed: May 2, 2018

(51) Int. Cl. A61F 13/20

(52) U.S. Cl. CPC A61F 13/2008; A61F 13/2065; A61F 13/2034; A61F 13/2074; A61F 13/2082; A61F 13/2068

(57) ABSTRACT

The present disclosure relates to fluid absorbers, along with a process for making the fluid absorber, which may be used, for example, by Parkinson's disease patients. The fluid absorber may include individual units that are folded into each other. The fluid absorber may be folded into a rectangle or square. The fluid absorber is then placed into an antimicrobial solution, and left to dry. In this way, a fluid absorber may have antimicrobial properties that reduce or eliminate tooth decay associated with hypersalivation. The fluid absorber is then placed inside the patient's mouth where it absorbs saliva until it becomes saturated. The fluid absorber may then be disposed.

### FIELD

This disclosure relates to the field of liquid absorption, and, in particular, the absorption of saliva in patients experiencing excessive drooling.

### BACKGROUND

The disclosure relates to a fluid absorber, and methods of making a fluid absorber.

There is interest in the manufacture of fluid absorbers that are operable and easy to assemble. There is also interest in providing a fluid absorber as an aid in treating disease and removing fluids, such as saliva. This is particularly true for individuals having Parkinson's disease, because Parkinson's disease may cause uncontrollable hypersalivation and drooling.

Hypersalivation is undesirable, because it may cause patients to stop taking their medications for Parkinson's disease, due to embarrassment and stigmatization. Hypersalivation may also lead to a condition called sialorrhea that causes patients to suffer from caries, aspiration pneumonia and skin maceration. Therefore, it would be advantageous to provide a method or apparatus for managing saliva in Parkinson's disease patients. The present inventions are intended to be used as supplements to other treatments.

Embodiments of the inventions overcome shortcomings in the prior art. Specifically, the prior art attempts to address the problem by providing medications which the patient ingests orally. However, these medications are costly. Disadvantageously, further side effects include draining the mouth of excess saliva and liquids from the patient's eyes and other body parts. This may cause discomfort, and, as mentioned above, may lead to further dental problems such as caries.

Accordingly, there is a need for new and improved methods and devices for managing saliva in Parkinson's disease patients. It would be advantageous to provide, for example, an absorption pad that is easily made or manufactured, which helps prevent caries.

### SUMMARY

These, as well as other aspects, advantages and alternatives, will become apparent to those of ordinary skill in the art by reading the following detailed description, with reference, where appropriate, to the accompanying drawings.

Systems and methods here include a fluid absorber, and methods for making and using such a fluid absorber. The fluid absorber may include individual units that are folded into each other. The fluid absorber may then be folded into a rectangle or square. The fluid absorber is placed into an absorbent solution, and left to dry. The fluid absorber is then placed inside the mouth.

Exemplary embodiments include a fluid absorber, with a plurality of individual units connected to each other. The plurality of individual squares may be folded into each other to form a plurality of grooves, in order increase absorption of fluids and surface area for the fluid absorber. Finally, an antimicrobial solution is provided to prevent caries and other dental problems.

Accordingly, it is an object of the present inventions to provide a method of manufacturing a fluid absorber by first folding a plurality of individual units into each other. Then, a plurality of grooves is formed through a plurality of folds in the individual units. The grooves are formed to increase surface area and absorption of the fluid absorber. The fluid absorber is then submerged in an antimicrobial solution. Finally, the fluid absorber is removed from the antimicrobial solution, and dried.

An additional object of the present inventions is to provide another method of manufacturing a fluid absorber by first folding a plurality of individual units into each other. Then, a plurality of grooves forms by folding adjacent members of the plurality of individual units. The grooves are formed to increase the surface area and absorption of the fluid absorber. Finally, an antimicrobial solution is embedded into the fluid absorber.

### BRIEF DESCRIPTION OF THE DRAWINGS

The embodiments will be more readily understood in view of the following description when accompanied by the below figures, wherein like numerals reference and represent like elements, wherein:

FIG. 1 is a view of a fluid absorber comprised of individual squares that can be folded into each other to form the fluid absorber or a first fold, according to an example embodiment.

FIG. 2A is a close-up view of the fluid absorber inside a patient's mouth and placed behind the teeth, according to an example embodiment.

FIG. 2B is another close-up view of the fluid absorber, placed below the tongue.

FIG. 2C is another close-up view of the fluid absorber, placed on the teeth.

FIG. 2D is another close-up view of the fluid absorber, placed between the teeth and gums.

FIG. 3 is a view of a patient holding the fluid absorber before inserting the fluid absorber into the mouth, according to an example embodiment.

FIG. 4 is a view of the fluid absorber placed on the teeth, according to an example embodiment.

FIG. 5 is a view of the fluid absorber placed near the front of the patient's lips and teeth, according to an example embodiment.

### DETAILED DESCRIPTION OF THE PREFERRED EMBODIMENTS

Reference will now be made in detail to embodiments, examples of which are illustrated in the accompanying drawings. In the following detailed description, numerous specific details are set forth in order to provide a sufficient understanding of the subject matter presented herein. But it will be apparent to one of ordinary skill in the art that the subject matter may be practiced without these specific details. Moreover, the particular embodiments described herein are provided by way of example and should not be used to limit the scope of the invention to these particular embodiments.

The present inventions include a fluid absorber that provides advantages over the prior art. The present inventions further include methods for making the same. Embodiments of the inventions include a fluid absorber 12 which may be placed in a variety of positions inside a patient's mouth for absorbing excessive saliva. The fluid absorber 12 may be made of absorbent materials such as paper, sponge, or cloth, and an antimicrobial solution such as mouthwash.

In certain exemplary embodiments, a patient 19 may use fluid absorber 12 by folding the fluid absorber 12 into itself. In certain example embodiments, alternatively or additionally, a patient may fold fluid absorber 12 into a rectangular compartment. In certain embodiments, portions of the fluid absorber 12 may collapse, and be folded into a square compartment. The square and rectangular compartments for the fluid absorber 12 are advantageous, because a patient can fold the fluid absorber 12 into a compact shape that allows for maximum absorption of saliva and other fluids. In certain embodiments, strips from the fluid absorber 12 may be torn to create a fluid absorber 12 with varying thicknesses. By having different thicknesses for the fluid absorber 12, patients can prevent excessive fluid absorption and tailor their hypersalivation treatments.

The present inventions may further include a method where the individual squares 14 of the fluid absorber 12 are folded into a square or rectangular unit. In this way, a patient may store and transport fluid absorber 12. Once a patient is ready to use fluid absorber 12, the assembly of fluid absorber 12 starts by folding an individual unit 14 into another. Each individual unit 14 is then folded until all the individual units 14 are completely folded to form a first fold 18. The first fold 18 is then folded again to form the fluid absorber 12. The fluid absorber 12 is dipped into an antimicrobial solution, such as mouthwash, and left to dry. The fluid absorber 12 is then placed in a mouth 24. In other embodiments, the size of the fluid absorber 12 can change by completing a different number of folds for the individual squares 14. Completing a different number of folds for the individual squares 14 enables patients to form a fluid absorber 12 that fits in the front and rear quadrants of their teeth, and allows patients 19 to tailor the fluid absorber 12's size and thickness.

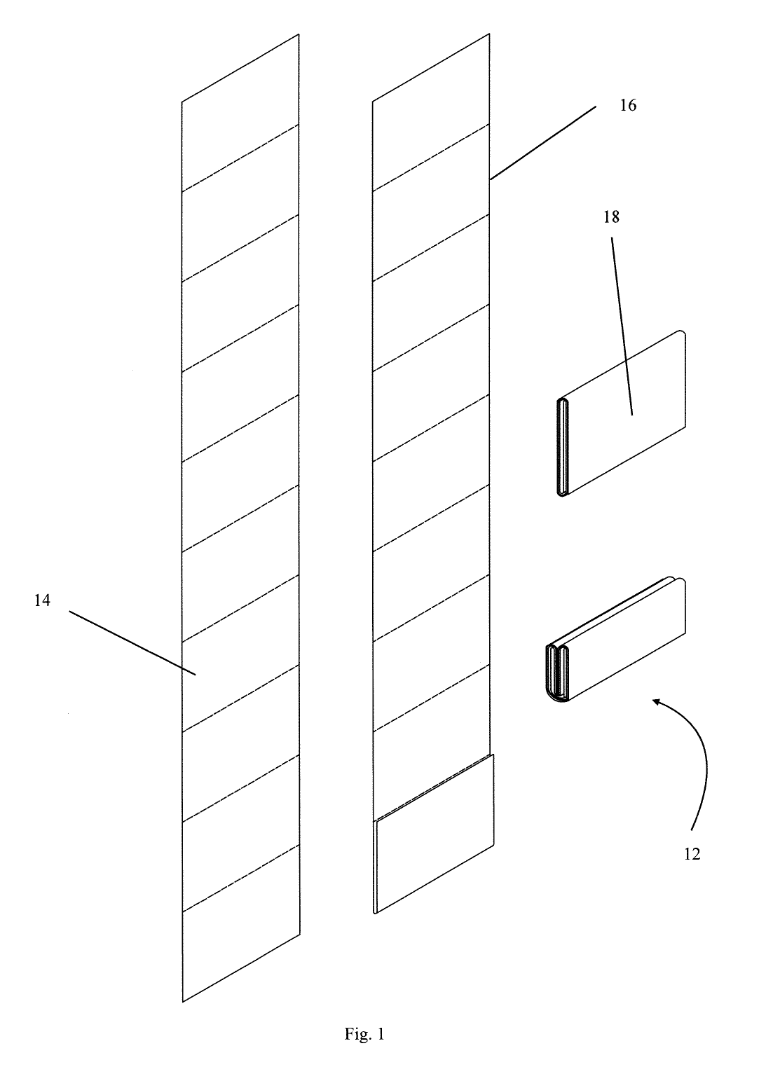

In an exemplary embodiment, a representative fluid absorber 12 is shown in FIG. 1. The terms fluid absorber, absorber, absorption pad, or other terms may be used interchangeably in the disclosure. They are not intended to be limiting but merely helpful to explain the ideas presented.

Referring to FIG. 1, an example fluid absorber 12 is shown in a folded or rolled configuration. The fluid absorber 12 may be configured to allow for maximum absorbance of fluids. It is known that an increased surface area leads to higher absorption. For this reason, embodiments of the invention provide a fluid absorber 12 that is comprised of multiple individual squares 14 that are folded. The grooves between the folds help increase surface area, and, by extension, absorption capacity.

The fluid absorber 12 can be made of any kind of absorbent material including, but not limited to, paper, sponge, cloth, wood or any other lightweight material. In preferred embodiments, the fluid absorber 12 may be made of an individual square 14 which may be arranged to fold into the fluid absorber 12. The individual square 14 may also be used to absorb fluids, as disclosed below. In a preferred embodiment, the size of the fluid absorber 12 has a width of 0.375 inches, a height of 0.25 inches and 1.75 to 2.0 inches in length.

In some embodiments, the individual square 14 is a component of the collapsed fluid absorber 16. Each individual square 14 may be folded into another individual square 14. Each individual square 14 may be folded from the collapsed fluid absorber 16, until all the individual squares 14 are folded to form the first fold 18.

In embodiments of the inventions, the fluid absorber 12 contains an antimicrobial solution. Including antimicrobial solution has the benefit of preventing caries and periodontal disease, reducing inflammation and sores in the mouth and aiding in treatments after surgery. In such embodiments, the fluid absorber 12 is placed in an antimicrobial solution such as mouthwash (not shown) for 1 hour. In an embodiment, the fluid absorber 12 rests in the antimicrobial solution (not shown) for 1 hour. During this time, the antimicrobial solution is absorbed into the fluid absorber 12. The fluid absorber 12 is then left to dry for up to 24 hours. After the drying period, a small amount of the antimicrobial solution remains in the fluid absorber 12 in order to retain and provide the benefits of the antimicrobial solution described above. In other embodiments, the fluid absorber 12 is made with an embedded antimicrobial solution. In some embodiments, the first fold 18 is folded again to form the fluid absorber 12. In other embodiments, the fluid absorber 12 may not contain any individual squares 14, a collapsed fluid absorber 16 or a first fold 18.

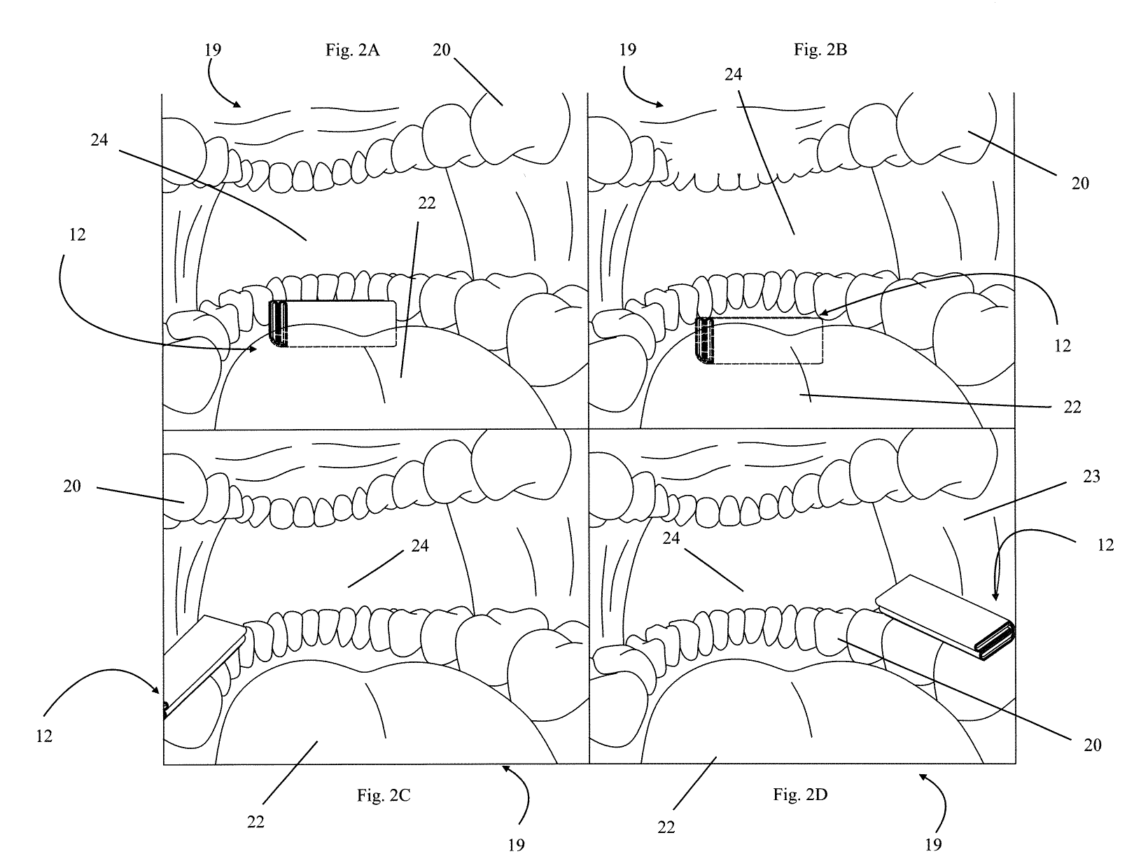

Referring now to FIG. 2A, a close-up view of the fluid absorber 12 inside a mouth 24 of a patient 19 is shown in an example embodiment. Fluids will pool or collect in lower quadrants of the mouth 24 without the fluid absorber 12, and will leak out of the mouth 24. To use the fluid absorber 12, a patient may place it behind teeth 20 in a front or rear quadrant of the mouth 24 and have the fluid absorber 12 partially covered by a tongue 22. The fluid absorber 12 will then collect fluids, particularly saliva (not shown). It is estimated that the fluid absorber 12 will become saturated after 4 to 5 hours, at which time the patient may replace the fluid absorber 12 with a new one. In another embodiment, saliva may collect in the patient 19's mouth 24 and the patient can sense the fluid absorber 12 collect fluids. The patient 19 may then depress the fluid absorber 12 between the teeth 20 to eliminate excess fluids. The patient 19 then swallows the excess fluids. Used fluid absorbers 12 may be thrown in the trash or otherwise discarded when the patient 19 removes the fluid absorber 12 from the mouth 20.

Referring now to FIG. 2B, another close-up view of the fluid absorber 12 inside the mouth 24 of the patient 19 is shown in an example embodiment. The tongue 22 covers the fluid absorber 12, and does not touch the teeth 20. The fluid absorber 12 will then collect fluids, particularly saliva (not shown). It is estimated that the fluid absorber 12 will become saturated after 4 to 5 hours, at which time the patient may replace the fluid absorber 12 with a new one. In another embodiment, saliva may collect in the patient 19's mouth 24 and the patient can sense the fluid absorber 12 collect fluids. The patient 19 may then depress the fluid absorber 12 between the teeth 20 to eliminate excess fluids. The patient 19 then swallows the excess fluids. Used fluid absorbers 12 may be thrown in the trash or otherwise discarded when the patient 19 removes the fluid absorber 12 from the mouth 20.

Referring now to FIG. 2C, another close-up view of the fluid absorber 12 inside the mouth 24 of the patient 19 is shown in an example embodiment. The fluid absorber 12 is placed on the teeth 20. The fluid absorber 12 also does not touch the tongue 22. The fluid absorber 12 will then collect fluids, particularly saliva (not shown). It is estimated that the fluid absorber 12 will become saturated after 4 to 5 hours, at which time the patient may replace the fluid absorber 12 with a new one. In another embodiment, saliva may collect in the patient 19's mouth 24 and the patient can sense the fluid absorber 12 collect fluids. The patient 19 may then depress the fluid absorber 12 between the teeth 20 to eliminate excess fluids. The patient 19 then swallows the excess fluids. Used fluid absorbers 12 may be thrown in the trash or otherwise discarded when the patient 19 removes the fluid absorber 12 from the mouth 20.

Referring now to FIG. 2D, another close-up view of the fluid absorber 12 inside the mouth 24 of the patient 19 and on the teeth 20 is shown in an example embodiment. The fluid absorber 12 does not touch the tongue 22. In another embodiment, the fluid absorber 12 is placed between the teeth 20 and gums 23. The fluid absorber 12 will then collect fluids, particularly saliva (not shown). It is estimated that the fluid absorber 12 will become saturated after 4 to 5 hours, at which time the patient may replace the fluid absorber 12 with a new one. In another embodiment, saliva may collect in the patient 19's mouth 24 and the patient can sense the fluid absorber 12 collect fluids. The patient 19 may then depress the fluid absorber 12 between the teeth 20 to eliminate excess fluids. The patient 19 then swallows the excess fluids. Used fluid absorbers 12 may be thrown in the trash or otherwise discarded when the patient 19 removes the fluid absorber 12 from the mouth 20.

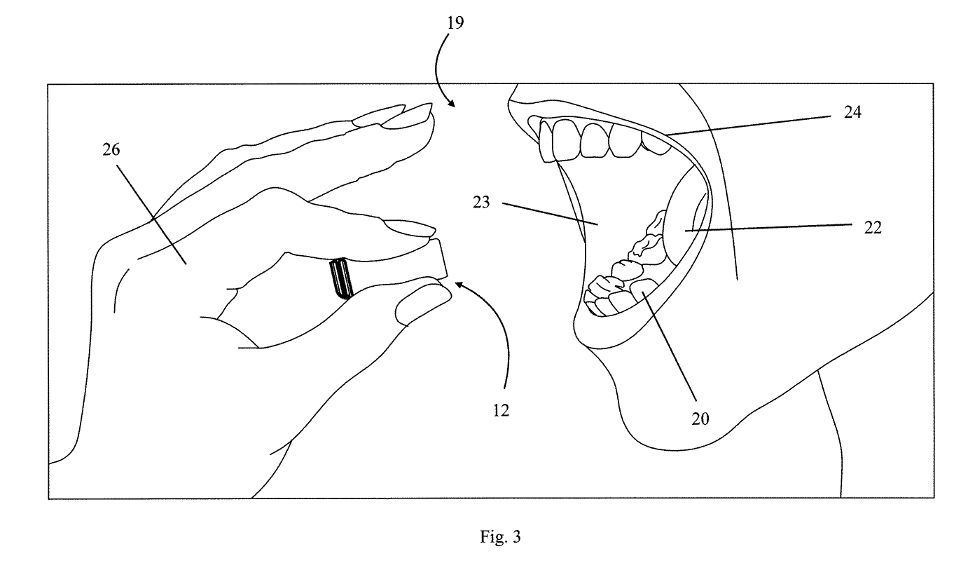

Referring now to FIG. 3, a view of the patient 19 inserting the fluid absorber 12 into the mouth 24 is shown. Once the fluid absorber 12 is dry, the patient 19 may grab the fluid absorber 12 with a hand 26. The hand 26 inserts the fluid absorber 12 into the mouth 24. As seen in FIGS. 2A-2D, the hand 26 may then place the fluid absorber 12 between the teeth 20 and tongue 22, under the tongue 22, on the teeth 20, or between the teeth 20 and the gums 23.

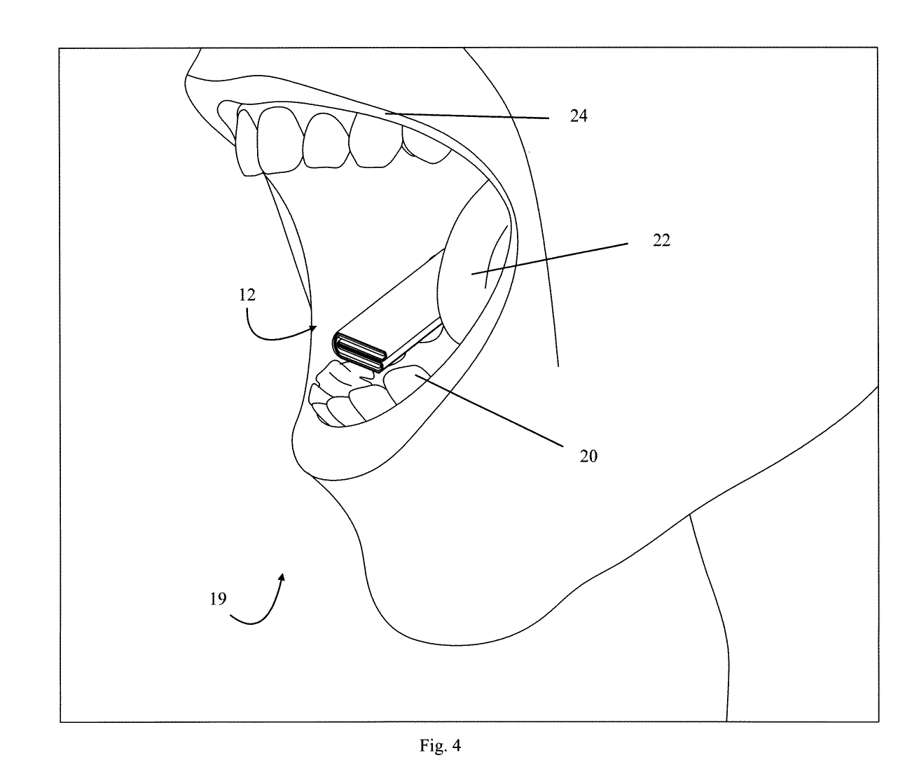

Referring now to FIG. 4, a view of the fluid absorber 12 inside the mouth 24 and on the teeth 20 is shown in an example embodiment. In one embodiment, the fluid absorber 12 may rest on the teeth 20. The hand 26 (not shown) may place the fluid absorber 12 in between the patient 19's teeth 20 and the gums 23, as seen in FIG. 2D. In one embodiment, the tongue 22 may touch the fluid absorber 12. In another embodiment, the tongue 22 neither touches the fluid absorber 12 nor the teeth 20.

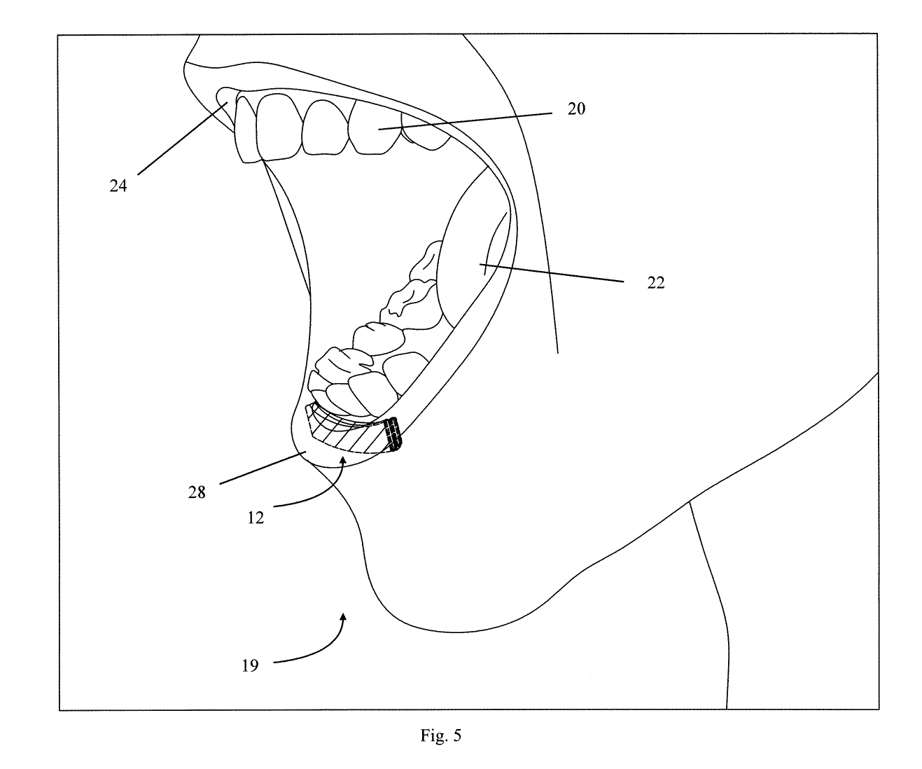

Referring now to FIG. 5, a view of the fluid absorber 12 placed underneath the teeth 20 and near lips 28 in an example embodiment is shown. The hand 26 (not shown) may place the fluid absorber 12 directly between the teeth 20 and the lips 28. In one embodiment, the tongue 22 may touch the fluid absorber 12. In another embodiment, the tongue 22 does not touch the fluid absorber 12 and the teeth 20.

The foregoing description, for purpose of explanation, has been described with reference to specific embodiments. However, the illustrative discussions above are not intended to be exhaustive or to limit the embodiments to the precise forms disclosed. Many modifications and variations are possible in view of the above teachings. The embodiments were chosen and described in order to best explain the principles of the embodiments and its practical applications, to thereby enable others skilled in the art to best utilize the various embodiments with various modifications as are suited to the particular use contemplated.

Unless the context clearly requires otherwise, throughout the description, the words “comprise,” “comprising,” and the like are to be construed in an inclusive sense as opposed to an exclusive or exhaustive sense; that is to say, in a sense of “including, but not limited to.” Words using the singular or plural number also include the plural or singular number respectively. Additionally, the words “herein,” “hereunder,” “above,” “below,” and words of similar import refer to this application as a whole and not to any particular portions of this application. When the word “or” is used in reference to a list of two or more items, that word covers all of the following interpretations of the word: any of the items in the list, all of the items in the list and any combination of the items in the list.

Although certain presently preferred implementations of the embodiments have been specifically described herein, it will be apparent to those skilled in the art to which the embodiments pertains that variations and modifications of the various implementations shown and described herein may be made without departing from the spirit and scope of the embodiments. Accordingly, it is intended that the embodiments be limited only to the extent required by the applicable rules of law.

What is claimed is:

1. A fluid absorber, comprising:
a plurality of individual units that are connected,
wherein the plurality of individual units may be folded into adjacent members of the plurality of individual units;
a thickness of the fluid absorber, determined by a number of folds generated by the plurality of individual units;
a plurality of grooves to increase absorption of fluids and surface area of the fluid absorber; and
an antimicrobial solution.

2. The fluid absorber of claim 1, wherein the plurality of individual units is comprised of paper, sponge, gauze or cloth.

3. The fluid absorber of claim 1, wherein the thickness of the fluid absorber increases as the number of folds for the plurality of individual units increases.

4. The fluid absorber of claim 3, wherein an amount of a fluid is absorbed, based on the thickness of the fluid absorber.

5. The fluid absorber of claim 1, wherein a length of the fluid absorber is 1.75 inches to 2 inches, a width of the fluid absorber is 0.375 inches and a height of the fluid absorber is 0.25 inches.

6. The fluid absorber of claim 1, wherein the plurality of individual units may be folded into a square or rectangular shape.

7. The fluid absorber of claim 1, wherein the plurality of grooves are located between each individual unit in the plurality of individual units.

8. The fluid absorber of claim 1, wherein the antimicrobial solution may be a mouthwash or other solution suitable for human ingestion.

9. The fluid absorber of claim 8, wherein the antimicrobial solution may be embedded into the fluid absorber.

10. The fluid absorber of claim 8, wherein the fluid absorber may be submerged in the antimicrobial solution for 1 hour.

11. The fluid absorber of claim 1, wherein the fluids the fluid absorber absorbs are comprised of saliva, blood, or mucus.

12. A method of manufacturing a fluid absorber, comprising:
folding a plurality of individual units into each other;
forming a plurality of grooves by folding adjacent members of the plurality of individual units together;
submerging the fluid absorber in an antimicrobial solution;
removing the fluid absorber from the antimicrobial solution, and drying the fluid absorber.

13. The method of claim 12, wherein the antimicrobial solution is a mouthwash or other solution suitable for human ingestion.

14. The method of claim 13, wherein the fluid absorber is submerged in the antimicrobial solution for 1 hour.

15. The method of claim 14, wherein the fluid absorber is dried for up to 24 hours.

16. The method of claim 12, wherein the antimicrobial solution remains in the fluid absorber after the fluid absorber is fully dried.

17. The method of claim 12, wherein a thickness of the fluid absorber is determined by a number of the plurality of grooves.

18. The method of claim 17, wherein the number of the plurality of grooves determines the surface area and rate of absorption of the fluid absorber.

19. The method of claim 12, wherein the plurality of individual units is comprised of paper, sponge, gauze or cloth.

20. A method of manufacturing a fluid absorber, comprising:
folding a plurality of individual units into each other;
forming a plurality of grooves by folding adjacent members of the plurality of individual units to increase surface area and absorption of the fluid absorber; and
embedding an antimicrobial solution into the fluid absorber.
